(19) United States
(12) Patent Application Publication
(10) Pub. No.: US 2019/0091127 A1
(43) Pub. Date: Mar. 28, 2019

(54) AGENTS AND METHODS FOR THE TEMPORARY SHAPING OF KERATIN-CONTAINING FIBERS

(71) Applicant: Henkel AG & Co. KGaA, Duesseldorf (DE)

(72) Inventors: Julia Bibiane Lange, Bad Bramstedt (DE); Anna Puls, Winsen (Luhe) (DE); Cyrielle Martinez, Hamburg (DE); Bernd Richters, Hamburg (DE)

(73) Assignee: Henkel AG & Co. KGaA, Duesseldorf (DE)

(21) Appl. No.: 15/527,097

(22) PCT Filed: Oct. 6, 2015
(86) PCT No.: PCT/EP2015/072991

§ 371 Date: May 16, 2017

(30) Foreign Application Priority Data
Nov. 17, 2014 (DE) 10 2014 223 398.4

(51) Int. Cl. A61K 8/81; A61Q 5/06; A61K 8/87

(52) U.S. Cl. CPC A61K 8/8147; A61K 8/87; A61Q 5/06

(57) ABSTRACT

A cosmetic agent for temporarily reshaping keratin fibers and a method for the same are provided herein. In one embodiment, the cosmetic agent includes, based on the total weight thereof, about 0.1 to about 2.0% by weight of at least one hydrophobically modified (meth)acrylic acid copolymer. The cosmetic agent includes, based on the total weight thereof, about 0.1 to about 6.0% by weight of at least one polyurethane/polyacrylate hybrid polymer. In another embodiment, the method includes applying a cosmetic agent to the keratin fibers. The cosmetic agent includes, based on the total weight thereof, about 0.1 to about 2.0% by weight of at least one hydrophobically modified (meth)acrylic acid copolymer. The cosmetic agent includes, based on the total weight thereof, about 0.1 to about 6.0% by weight of at least one polyurethane/polyacrylate hybrid polymer. The method includes temporarily imparting hold to the shape of the keratin-containing fibers.

### CROSS-REFERENCE TO RELATED APPLICATION

This application is a U.S. National-Stage entry under 35 U.S.C. § 371 based on International Application No. PCT/EP2015/072991, filed Oct. 6, 2015 which was published under PCT Article 21(2) and which claims priority to German Application No. 10 2014 223 398.4, filed Nov. 17, 2014, which are all hereby incorporated in their entirety by reference.

### TECHNICAL FIELD

This application pertains to a cosmetic agent for fixing hair or for temporarily reshaping keratin fibers, in particular human hair, wherein the agent comprises a combination of two polymers.

### BACKGROUND

Temporarily creating hair styles for an extended period of up to several days generally requires the use of fixative active ingredients. Hair treatment agents that are used to temporarily impart shape to the hair therefore play an important role. Corresponding agents for temporary reshaping usually comprise synthetic polymers and/or waxes serving as the fixative active ingredient. Agents for supporting the temporary shaping of keratin-containing fibers can be formulated in the form of hair spray, hair wax, hair gel, or hair mousse, for example.

The most important property of an agent for temporarily reshaping hair, hereafter also referred to as a styling agent, is to give the treated fibers the strongest hold possible in the newly modeled shape, which is to say a shape that has been imparted to the hair. This is also referred to as strong styling hold or a high degree of hold of the styling agent. The styling hold is essentially determined by the nature and amount of the fixative active ingredient that is used, although further components of the styling agent may also have an influence

In addition to a high degree of hold, styling agents must satisfy a whole host of additional requirements. These can be broken down in approximate terms into properties of the hair, properties of the individual formulation, such as properties of the mousse, of the gel, or of the sprayed aerosol, and properties that relate to the handling of the styling agent, wherein the properties of the hair are particularly important. In particular, moisture resistance, strength, low tack, and a balanced conditioning effect shall be mentioned. Moreover, a styling agent should be universally suitable for all hair types to an extent as great as possible, and be gentle on the hair and skin.

In order to meet the diverse requirements, a number of synthetic polymers have already been developed as fixative active ingredients, which are used in styling agents. The polymers can be divided into cationic, anionic, non-ionic and amphoteric fixative polymers. Ideally, the polymers form a polymer film when applied to the hair, which gives the hair style a strong hold on the one hand, but on the other hand is sufficiently flexible so as not to break under stress.

### BRIEF SUMMARY

A cosmetic agent for temporarily reshaping keratin fibers and a method for the same are provided herein. In one embodiment, the cosmetic agent includes, based on the total weight thereof, about 0.1 to about 2.0% by weight of at least one hydrophobically modified (meth)acrylic acid copolymer. The cosmetic agent includes, based on the total weight thereof, about 0.1 to about 6.0% by weight of at least one polyurethane/polyacrylate hybrid polymer.

In another embodiment, the method includes applying a cosmetic agent to the keratin fibers. The cosmetic agent includes, based on the total weight thereof, about 0.1 to about 2.0% by weight of at least one hydrophobically modified (meth)acrylic acid copolymer. The cosmetic agent includes, based on the total weight thereof, about 0.1 to about 6.0% by weight of at least one polyurethane/polyacrylate hybrid polymer. The method includes temporarily imparting hold to the shape of the keratin-containing fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

An exemplary styling agent having good hold properties and high moisture resistance, which is based on a combination of a hydrophobically modified (meth)acrylic acid copolymer and a hydrophobically modified polysaccharide, is described in German patent application DE 10 2012 214 380 A1.

Hair gels comprising hybrid polymers of polyurethane and polymethyl methacrylate as one component are described in European patent applications EP 2286908 A2 and EP 2455157 A1. International patent application WO2014012918 A2 describes aqueous dispersions of polyurethane/polyacrylate hybrid polymers as a component of styling agents.

It was an object of the present disclosure to provide further suitable polymer combinations, which are characterized by good film-forming and/or fixative properties, have a high degree of hold, without having to forgo flexibility and good moisture resistance, and in particular perspiration and water resistance, and are also suitable for producing cosmetic compositions having stable viscosity, and preferably stable transparency. In particular, presently available styling agents still offer room for improvement in that a good combination of stiffness and long-lasting hold is not always sufficiently ensured. It is therefore the object of the present disclosure to provide styling agents that, in addition to the above-mentioned properties, result in particular in good stiffness and good long-lasting hold.

This object was able to be achieved by the combination of two specific polymers. A further subject matter of the present application is thus a cosmetic agent for temporarily reshaping keratin fibers, comprising, based on the total weight thereof:
a) about 0.1 to about 2.0% by weight of at least one hydrophobically modified (meth)acrylic acid copolymer;
b) about 0.1 to about 6.0% by weight of at least one polyurethane/polyacrylate hybrid polymer.

A first essential component of cosmetic agents as contemplated herein is the hydrophobically modified (meth)acrylic acid copolymer a). Preferred copolymers a) have a thickening effect. A percentage by weight of copolymer a) relative to the total weight of the cosmetic agent of about 0.2 to about 1.5% by weight, preferably about 0.3 to about 1.0% by weight, and in particular about 0.3 to about 0.7% by weight has proven advantageous for the cosmetic action.

The hydrophobically modified (meth)acrylic acid copolymers a) used are preferably copolymers that can be traced back to

at least one monomer (a1) from the group of the unsaturated carboxylic acids and unsaturated carboxylic acid esters, and

at least one monomer (a2) from the group of the unsaturated hydrophobically modified monomers.

Preferred copolymers a) are based on at least one monomer (a1) from the group consisting of acrylic acid, methacrylic acid, C₁-C₆ alkyl acrylic acid esters, and C₁-C₆ alkyl methacrylic acid esters. The acrylic acid esters and methacrylic acid esters are preferably esters of the respective acids with non-tertiary alkanols with alkyl residues of 1 to 12 carbon atoms, and in particular 2 to 4 carbon atoms. For example, ethyl acrylate, ethyl methacrylate, propyl acrylate, propyl methacrylate, n-butyl acrylate, n-butyl methacrylate, isobutyl acrylate, 2-methyl butylacrylate, 2-ethylhexyl acrylate, n-octyl acrylate, isooctyl acrylate, isooctyl methacrylate, isononyl acrylate and isodecyl acrylate shall be mentioned as suitable monomers.

The group of hydrophobically modified monomers (a2) denotes monomers that have a hydrophobic sub-structure. Preferred monomers (a2) can be traced back to the following two structural units:

an unsaturated acid, preferably acrylic acid, methacrylic acid or itaconic acid;

a C₈₋₄₀ alkyl chain, preferably a C₁₀₋₃₀ alkyl chain.

These two sub-structures can optionally be supplemented with a third structural unit from the group of the polyoxyalkylene groups, and preferably the polyethylene glycol groups, the polypropylene glycol groups or the polyethylene glycol/polypropylene glycol groups.

For example, C₁₀₋₃₀ alkyl acrylates, C₁₀₋₃₀ alkyl PEG acrylates, C₁₀₋₃₀ alkyl PEG methacrylates or C₁₀₋₃₀ alkyl PEG itaconates are used as the monomer (a2). Preferred monomers (a2) are selected from the C₁₀₋₃₀ alkyl acrylates, C₁₀₋₃₀ alkyl PEG 20-25 acrylates, C₁₀₋₃₀ alkyl PEG 20-25 methacrylates or C₁₀₋₃₀ alkyl PEG 20-25 itaconates. Particularly preferred monomers (a2) are selected from the group consisting of C₁₀₋₃₀ alkyl acrylates, Steareth-20 methacrylates, Beheneth-25 methacrylates, Steareth-20 itaconates, Ceteth-20 itaconates, Palmeth-25 acrylates or C₁₀₋₃₀ alkyl PEG-20 itaconates.

In summary, preferred copolymers a) are those formed of

at least one monomer (a1) from the group consisting of acrylic acid, methacrylic acid, C₁-C₆ alkyl acrylic acid esters, and C₁-C₆ alkyl methacrylic acid esters;

at least one monomer (a2) from the group consisting of the C₁₀₋₃₀ alkyl acrylates, C₁₀₋₃₀ alkyl PEG acrylates, C₁₀₋₃₀ alkyl PEG methacrylates or C₁₀₋₃₀ alkyl PEG itaconates.

Particularly preferred copolymers a) are those obtained by reacting

at least one monomer (a1) from the group consisting of acrylic acid, methacrylic acid, C₁-C₆ alkyl acrylic acid esters, and C₁-C₆ alkyl methacrylic acid esters with

at least one monomer (a2) from the group consisting of the C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates.

In addition to the above-described monomers (a1) and (a2), further preferred hydrophobically modified (meth)acrylic acid copolymers a) are formed of at least one monomer (a3) from the group of the unsaturated amine group-containing monomers.

Preferably, monomers from the group consisting of acrylamide, methacrylamide, mono-(C₁-C₄)-alkylamino(C₁-C₄) alkyl acrylate, di-(C₁-C₄)-alkylamino(C₁-C₄)-alkyl acrylate, mono-(C₁-C₄)-alkylamino(C₁-C₄)-alkyl methacrylate, di-(C₁-C₄)-alkylamino(C₁-C₄)-alkyl methacrylate are used as monomer (a3). 2-(N,N-dimethylamino)ethyl acrylat, 2-(N,N-dimethylamino)ethyl methacrylates, 2-(N,N-diethylamino)ethyl acrylates, 2-(N,N-diethylamino)ethyl methacrylates, 3-(N,N-dimethylamino)propyl acrylate, 3-(N,N-dimethylamino)propyl methacrylate, 2-(N,N-dimethylamino)neopentyl acrylate, N′-(3-N,N-dimethylamino)propyl acrylamide, N′-(3-N,N-dimethylamino)propyl methacrylamide are exemplary and preferred substances used as monomer (a3).

Preferred copolymers a) are those formed of

at least one monomer (a1) from the group consisting of acrylic acid, methacrylic acid, C₁-C₆ alkyl acrylic acid esters, and C₁-C₆ alkyl methacrylic acid esters;

at least one monomer (a2) from the group of the C₁₀₋₃₀ alkyl PEG 20-itaconates;

at least one monomer (a3) from the group consisting of acrylamide, methacrylamide, mono-(C₁-C₄)-alkylamino(C₁-C₄)-alkyl acrylate, di-(C₁-C₄)-alkylamino(C₁-C₄)-alkyl acrylate, mono-(C₁-C₄)-alkylamino(C₁-C₄)-alkyl methacrylate, and di-(C₁-C₄)-alkylamino(C₁-C₄)-alkyl methacrylate.

Particularly preferably used copolymers a) are cross-linked.

In summary, preferred cosmetic agents as contemplated herein are characterized in that copolymer a) is selected from the group consisting of the compounds bearing the INCI names Acrylates/C₁₀₋₃₀-Alkyl Methacrylate Copolymer, Acrylates/C₁₀₋₃₀-Alkyl Acrylate Crosspolymer, Acrylates/Steareth-20 Methacrylate Crosspolymer, Acrylates/Steareth-20 Methacrylate Copolymer, Acrylates/Beheneth-25 Methacrylate Copolymer, Acrylates/Steareth-20 Itaconate Copolymer, Acrylates/Ceteth-20 Itaconate Copolymer, Acrylates/Palmeth-25 Acrylate Copolymer, Acrylates/Aminoacrylates/C₁₀₋₃₀ Alkyl PEG-20 Itaconate Copolymer. Corresponding polymers are available under the trade names Luvigel® FIT, Ultrez® 21 , Pemulen® TR1, Aculyn® 22, Aculyn® 28, Aculyn® 88, Structure® 2001 , Structure® 3001, Synthalen® W2000 and Structure® Plus, for example. Copolymer a) is particularly preferably selected from the group of the compounds bearing the INCI name Acrylates/C₁₀₋₃₀ Alkyl Methacrylate Crosspolymer.

The agents as contemplated herein comprise a hybrid polymer of polyurethane and polyacrylate as a second essential component. Examples of such copolymers, which are also referred to as interpenetrating polymer networks, are hybrid polymers of polyurethane and polymethyl methacrylate (see, for example, S. C. Kim et al., Macromolecules, 1976, 9(2), pp. 258-263). Preferably, the polyurethane/polyacrylate hybrid polymer (b) is used in the form of an aqueous dispersion. The percentage by weight of the polyurethane/polyacrylate hybrid polymer b) relative to the total weight of the cosmetic agent is especially about 0.2 to about 4.0% by weight, preferably about 0.4 to about 3.0% by weight, and in particular about 0.8 to about 2.0% by weight.

The polyurethane of the polyurethane/polyacrylate hybrid polymer (b) is preferably a copolymer of the monomers hexylene glycol, neopentyl glycol, adipic acid, methylene diphenyl diisocyanate and dimethylpropionic acid (INCI: Polyurethane-2).

It is furthermore preferred when the polyacrylate of the polyurethane/polyacrylate hybrid polymer (b) is a polymethyl methacrylate.

The polyurethane/polyacrylate hybrid polymer (b) is most preferably one bearing the INCI name Polyurethane-2 and Polymethyl Methacrylate.

An especially particularly preferred polyurethane/polyacrylate hybrid polymer (b) is referred to as Polyurethane-2 and Polymethyl Methacrylate according to INCI nomenclature. Such a polymer is available under the trade name Hybridur® 875 (Air Products) in the form of an approximately 40% by weight dispersion, for example (common properties: anionic, pH approximately 7.5 to 9.0, viscosity Brookfield cPs approximately 50 to 100, particle size approximately 75 to 80 nm).

In addition to the above-described advantages, the agents as contemplated herein in particular stand out among the cosmetic agents comprising alternative copolymers (b) by an improved degree of hold, low tack, and high stiffness. A weight ratio of polymers a) and b) in the cosmetic agent of about 1:1 to about 1:10, preferably about 1:2 to about 1:8, and in particular about 1:4 to about 1:6 has proven particularly advantageous for the cosmetic properties of the agents as contemplated herein.

Copolymer a) is preferably used in the cosmetic agent in a partially neutralized or neutralized form. Preferably, at least one alkanolamine is used for neutralization. The alkanolamines that can be used as alkalizing agents as contemplated herein are preferably selected from primary amines having a C₂-C₆ alkyl base body, which carries at least one hydroxyl group. Particularly preferred alkanolamines are selected from the group consisting of 2-aminoethane-1-ol (monoethanolamine), tris(2-hydroxyethyl)-amine (triethanolamine), 3-aminopropane-1-ol, 4-aminobutane-1-ol, -aminopentane-1-ol, 1-aminopropane-2-ol, 1-aminobutane-2-ol, 1-aminopentane-2-ol, 1-aminopentane-3-ol, 1-aminopentane-4-ol, 3-amino-2-methylpropane-1-ol, 1-amino-2-methylpropane-2-ol, 3-aminopropane-1,2-diol, 2-amino-2-methylpropane-1,3-diol. Especially particularly preferred alkanolamines as contemplated herein are selected from the group consisting of 2-aminoethane-1-ol, 2-amino-2-methylpropane-1-ol and 2-amino-2-methylpropane-1,3-diol. In this, 2-amino-2-methylpropanol has proven to be a particularly suited neutralizing agent. Preferred cosmetic agents as contemplated herein comprise at least one alkanolamine, and preferably 2-amino-2-methylpropanol. The 2-amino-2-methylpropanol is preferably used in the agents as contemplated herein in an amount that does not exceed the amount required for neutralizing copolymer a). The amount of 2-amino-2-methylpropanol used in the agents as contemplated herein is preferably about 80 to 100%, especially preferably about 90 to 100%, and in particular about 95 to 100% of the amount required to fully neutralize copolymer a). In a preferred embodiment, the percentage by weight of the 2-amino-2-methypropanol relative to the total weight of the cosmetic agent is about 0.01 to about 2.0% by weight, preferably about 0.01 to about 1.0% by weight, and in particular about 0.02 to about 0.5% by weight. Especially particularly preferred are percentages by weight of the amino alcohol, and preferably of the 2-amino-2-methylpropanol, of about 0.01 to about 0.2% by weight, preferably about 0.01 to about 0.1% by weight, and in particular about 0.02 to about 0.6% by weight.

In addition to the above-described copolymers a) and copolymers b), the cosmetic agents as contemplated herein can comprise further active ingredients, auxiliary substances and nourishing agents.

The film-forming polymers are a first group of preferably used active ingredients. These film-forming polymers are not identical to the above-described hydrophobically modified (meth)acrylic acid copolymer a) or the vinylpyrrolidone copolymers b). The percentage by weight of the film-forming polymer relative to the total weight of the cosmetic agents is especially about 0.1 to about 8.0% by weight, preferably about 0.5 to about 6.0% by weight, and in particular about 1.0 to about 4.0% by weight.

Non-ionic polymers are preferably used as film-forming polymers. Suitable non-ionic polymer are, for example:

vinylpyrrolidone/vinyl ester copolymers, as they are sold, for example, under the trade name Luviskol® (BASF). Luviskol® VA 64 and Luviskol® VA 73, each being vinylpyrrolidone/vinyl acetate copolymers, are preferred nonionic polymers.

cellulose ethers, such as hydroxypropyl cellulose, hydroxyethyl cellulose and hydroxypropyl methylcellulose, as they are sold, for example, under the trade names Culminal® and Benecel® (AQUALON);

shellac;

polyvinylpyrrolidones, as they are sold, for example, under the designation Luviskol® (BASF);

siloxanes. These siloxanes can be either water-soluble or water-insoluble. Both volatile and non-volatile siloxanes are suitable, wherein non-volatile siloxanes shall be understood to mean those compounds having a boiling point above 200° C. at normal pressure. Preferred siloxanes are polydialkylsiloxanes, such as polydimethylsiloxane, polyalkylarylsiloxanes, such as polyphenylmethylsiloxane, ethoxylated polydialkylsiloxanes, and polydialkylsiloxanes containing amine and/or hydroxy groups.

Glycosidically substituted silicones.

Given the cosmetic action thereof in combination with copolymers a) and b), film-forming polymers that are preferably used as contemplated herein are in particular the polyvinylpyrrolidones (INCI name: PVP) and the vinylpyrrolidone/vinyl acetate copolymers (INCI name: VP/VA Copolymer), wherein the percentage by weight of these polymers is preferably limited to amounts between about 1.0 and about 10% by weight. Particularly preferred cosmetic agents as contemplated herein are thus characterized by furthermore comprising, based on the total weight thereof, about 1.0 to about 10% by weight polyvinylpyrrolidone and/or vinylpyrrolidone/vinyl acetate copolymer, preferably polyvinylpyrrolidone. Particularly preferred cosmetic agents have a percentage by weight of the polyvinylpyrrolidone and/or vinylpyrrolidone/vinyl acetate copolymer c), relative to the total weight of the cosmetic agent, of about 2.0 to about 8.5% by weight, and preferably of about 3.0 to about 7.0% by weight.

In summary, particularly preferred cosmetic agents as contemplated herein comprise three different polymers, these being copolymers a) and b) and the film-forming polymer c). In a preferred embodiment, the cosmetic agents, based on the total weight thereof, comprise:
a) about 0.1 to about 2.0% by weight of at least one copolymer a), which is obtained by reacting

at least one monomer (a1) from the group consisting of acrylic acid, methacrylic acid, C₁-C₆ alkyl acrylic acid esters, and C₁-C₆ alkyl methacrylic acid esters with

at least one monomer (a2) from the group consisting of the C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates;
b) about 0.1 to about 6.0% by weight of at least one polyurethane/polyacrylate hybrid polymer;
c) about 1.0 to about 10% by weight polyvinylpyrrolidone.

Particularly preferred are cosmetic agents that, based on the total weight thereof, comprise:
a) about 0.3 to about 1.0% by weight of a copolymer a) from the group of the compounds bearing the INCI name Acrylates/C10-30 Alkyl Methacrylate Crosspolymer;
b) about 0.3 to about 1.0% by weight of a copolymer b) from the group of the compounds bearing the INCI name Polyurethane-2 and Polymethyl Methacrylate;
c) about 3.0 to about 7.0% by weight polyvinylpyrrolidone.

The nourishing agent used can be protein hydrolyzates and/or the derivatives thereof. Protein hydrolyzates are product mixtures that are obtained by the acidically, basically or enzymatically catalyzed degradation of proteins. The term ‘protein hydrolyzates’ as contemplated herein shall also be understood to cover total hydrolyzates and individual amino acids and the derivatives thereof, as well as mixtures of different amino acids. The molecular weight of the protein hydrolyzates that can be used as contemplated herein ranges between about 75, the molecular weight for glycine, and about 200,000; the molecular weight is preferably about 75 to about 50,000, and especially particularly preferably 75 to 20,000 daltons.

Vitamins, provitamins, vitamin precursors and/or the derivatives thereof are another group of nourishing agents. As contemplated herein, vitamins, provitamins and vitamin precursors that are usually assigned to groups A, B, C, E, F and H are preferred.

Glycerol, propylene glycol, panthenol, caffeine, nicotinamide and sorbitol are further nourishing agents.

It is also possible to use plant extracts, or monosaccharides or oligosaccharides and/or lipids as nourishing agents.

Preferred cosmetic agents are based on an aqueous or aqueous/alcoholic carrier. The water content, relative to the total weight of cosmetic agents as contemplated herein, is preferably at least about 10% by weight, in particular at least about 20.0% by weight, more preferably at least about 40% by weight, and particularly preferably at least about 70% by weight. The water content of cosmetic agents as contemplated herein is especially about 10 to about 98% by weight, preferably about 30 to about 98% by weight, particularly preferably about 50 to about 98% by weight, and in particular about 70 to about 97% by weight.

The compositions of several cosmetic agents used can be derived from the following tables (information in percent by weight is based on the total weight of the cosmetic agent, unless indicated otherwise).

| | Formula 1 | Formula 2 | Formula 3 | Formula 4 | Formula 5 |
|---|---|---|---|---|---|
| Copolymer a)¹⁾ | 0.1 to 2.0 | 0.2 to 1.5 | 0.3 to 1.0 | 0.3 to 1.0 | 0.3 to 0.7 |
| Copolymer b)²⁾ | 0.1 to 6.0 | 0.2 to 4.0 | 0.2 to 4.0 | 0.4 to 3.0 | 0.8 to 2.0 |
| Optional additives | to make up | to make up | to make | to make up | to make |
| | to 100 | to 100 | up to 100 | to 100 | up to 100 |

| | | | | | Formula |
|---|---|---|---|---|---|
| | Formula 6 | Formula 7 | Formula 8 | Formula 9 | 10 |
| Copolymer a)¹⁾ | 0.1 to 2.0 | 0.2 to 1.5 | 0.3 to 1.0 | 0.3 to 1.0 | 0.3 to 0.7 |
| Copolymer b)²⁾ | 0.1 to 6.0 | 0.2 to 4.0 | 0.2 to 4.0 | 0.4 to 3.0 | 0.8 to 2.0 |
| Water | 10 to 98 | 10 to 98 | 30 to 98 | 50 to 98 | 70 to 97 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 11 | Formula 12 | 13 | Formula 14 | 15 |
| Copolymer a)³⁾ | 0.1 to 2.0 | 0.2 to 1.5 | 0.3 to 1.0 | 0.3 to 1.0 | 0.4 to 0.6 |
| Copolymer b)²⁾ | 0.1 to 6.0 | 0.2 to 4.0 | 0.2 to 4.0 | 0.4 to 3.0 | 0.8 to 2.0 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 16 | Formula 17 | 18 | Formula 19 | 20 |
| Copolymer a)³⁾ | 0.1 to 2.0 | 0.2 to 1.5 | 0.3 to 1.0 | 0.3 to 1.0 | 0.3 to 0.7 |
| Copolymer b)²⁾ | 0.1 to 6.0 | 0.2 to 4.0 | 0.2 to 4.0 | 0.4 to 3.0 | 0.8 to 2.0 |
| Water | 10 to 98 | 10 to 98 | 30 to 98 | 50 to 98 | 70 to 97 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 21 | Formula 22 | 23 | Formula 24 | 25 |
| Copolymer a)¹⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 26 | Formula 27 | 28 | Formula 29 | 30 |
| Copolymer a)¹⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Water | 10 to 98 | 10 to 98 | 30 to 98 | 50 to 98 | 70 to 97 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 31 | Formula 32 | 33 | Formula 34 | 35 |
| Copolymer a)³⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 36 | Formula 37 | 38 | Formula 39 | 40 |
| Copolymer a)³⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Water | 10 to 98 | 10 to 98 | 30 to 98 | 50 to 98 | 70 to 97 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 41 | Formula 42 | 43 | Formula 44 | 45 |
| Acrylates/C₁₀₋₃₀ Alkyl | 0.1 to 2.0 | 0.2 to 1.5 | 0.3 to 1.0 | 0.3 to 1.0 | 0.3 to 0.7 |
| Methacrylate | | | | | |
| Crosspolymer | | | | | |
| Polyurethane-2 and | 0.1 to 6.0 | 0.2 to 4.0 | 0.2 to 4.0 | 0.4 to 3.0 | 0.8 to 2.0 |
| Polymethyl Methacrylate | | | | | |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 46 | Formula 47 | 48 | Formula 49 | 50 |
| Acrylates/C₁₀₋₃₀ Alkyl | 0.1 to 2.0 | 0.2 to 1.5 | 0.3 to 1.0 | 0.3 to 1.0 | 0.3 to 0.7 |
| Methacrylate | | | | | |
| Crosspolymer | | | | | |
| Polyurethane-2 and | 0.1 to 6.0 | 0.2 to 4.0 | 0.2 to 4.0 | 0.4 to 3.0 | 0.8 to 2.0 |
| Polymethyl Methacrylate | | | | | |
| Water | 10 to 98 | 10 to 98 | 30 to 98 | 50 to 98 | 70 to 97 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 51 | Formula 52 | 53 | Formula 54 | 55 |
| Copolymer a)¹⁾ | 0.1 to 2.0 | 0.2 to 1.5 | 0.3 to 1.0 | 0.3 to 1.0 | 0.3 to 0.7 |
| Copolymer b)²⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 56 | Formula 57 | 58 | Formula 59 | 60 |
| Copolymer a)¹⁾ | 0.1 to 2.0 | 0.2 to 1.5 | 0.3 to 1.0 | 0.3 to 1.0 | 0.3 to 0.7 |
| Copolymer b)²⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 61 | Formula 62 | 63 | Formula 64 | 65 |
| Copolymer a)³⁾ | 0.1 to 2.0 | 0.2 to 1.5 | 0.3 to 1.0 | 0.3 to 1.0 | 0.3 to 0.7 |
| Copolymer b)²⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 66 | Formula 67 | 68 | Formula 69 | 70 |
| Copolymer a)³⁾ | 0.1 to 2.0 | 0.2 to 1.5 | 0.3 to 1.0 | 0.3 to 1.0 | 0.3 to 0.7 |
| Copolymer b)²⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 71 | Formula 72 | 73 | Formula 74 | 75 |
| Copolymer a)¹⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 76 | Formula 77 | 78 | Formula 79 | 80 |
| Copolymer a)¹⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 81 | Formula 82 | 83 | Formula 84 | 95 |
| Copolymer a)³⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 86 | Formula 87 | 88 | Formula 89 | 90 |
| Copolymer a)³⁾ | 0.1 to 2.0 | 0.2 to 1.5 | 0.3 to 1.0 | 0.3 to 1.0 | 0.3 to 0.7 |
| Copolymer b)⁴⁾ | 0.1 to 6.0 | 0.2 to 4.0 | 0.2 to 4.0 | 0.4 to 3.0 | 0.8 to 2.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 91 | Formula 92 | 93 | Formula 94 | 95 |
| Acrylates/C₁₀₋₃₀ Alkyl | 0.1 to 2.0 | 0.2 to 1.5 | 0.3 to 1.0 | 0.3 to 1.0 | 0.3 to 0.7 |
| Methacrylate | | | | | |
| Crosspolymer | | | | | |
| Polyurethane-2 and | 0.1 to 6.0 | 0.2 to 4.0 | 0.2 to 4.0 | 0.4 to 3.0 | 0.8 to 2.0 |
| Polymethyl Methacrylate | | | | | |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to make up | to make up | to make | to make up | to make |
| | to 100 | to 100 | up to 100 | to 100 | up to 100 |

| | | | Formula | | Formula |
|---|---|---|---|---|---|
| | Formula 96 | Formula 97 | 98 | Formula 99 | 100 |
| Acrylates/C₁₀₋₃₀ Alkyl | 0.1 to 2.0 | 0.2 to 1.5 | 0.3 to 1.0 | 0.3 to 1.0 | 0.3 to 0.7 |
| Methacrylate | | | | | |
| Crosspolymer | | | | | |
| Polyurethane-2 and | 0.1 to 6.0 | 0.2 to 4.0 | 0.2 to 4.0 | 0.4 to 3.0 | 0.8 to 2.0 |
| Polymethyl Methacrylate | | | | | |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to make up | to make up | to make | to make up | to make up |
| | to 100 | to 100 | up to 100 | to 100 | to 100 |

¹) hydrophobically modified (meth)acrylic acid copolymer
²) polyurethane/polyacrylate hybrid polymer
³) copolymer a), which is obtained by reacting
at least one monomer (a1) from the group consisting of acrylic acid, methacrylic acid, C₁-C₆ alkyl acrylic acid esters, and C₁-C₆ alkyl methacrylic acid esters with
at least one monomer (a2) from the group consisting of the C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates
⁴) polyurethane-2-polyacrylate hybrid polymer

Preferred cosmetic agents are present in the form of hair gel, hair spray, hair mousse or hair wax.

As described at the outset, the above-described cosmetic agents are characterized by special properties as they relate to hair cosmetics, and in particular by advantageous properties for temporarily reshaping hair. A second subject matter of the present application is thus the use of an agent as contemplated herein for temporarily reshaping keratin-containing fibers, and in particular human hair.

A third subject matter of the present application is a method for temporarily reshaping keratin-containing fibers, and in particular human hair, in which a cosmetic agent as contemplated herein is applied to the keratin fibers and the shape of these is temporarily imparted hold.

The agents, uses and methods as contemplated herein and several of the preferred embodiments thereof are characterized by the following items:

1. A cosmetic agent for temporarily reshaping keratin fibers, comprising, based on the total weight thereof:
a) about 0.1 to about 2.0% by weight of at least one hydrophobically modified (meth)acrylic acid copolymer;
b) about 0.1 to about 6.0% by weight of at least one polyurethane/polyacrylate hybrid polymer.

2. The cosmetic agent according to item 1, characterized in that the percentage by weight of copolymer a), relative to the total weight of the cosmetic agent, is about 0.2 to about 1.5% by weight, preferably about 0.3 to about 1.0% by weight, and in particular about 0.3 to about 0.7% by weight.

3. The cosmetic agent according to any one of the preceding items, characterized in that copolymer a) is obtained by reacting

at least one monomer (a1) from the group consisting of acrylic acid, methacrylic acid, C₁-C₆ alkyl acrylic acid esters, and C₁-C₆ alkyl methacrylic acid esters with

at least one monomer (a2) from the group consisting of the C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates.

4. The cosmetic agent according to any one of the preceding items, characterized in that copolymer a) is selected from the group of the compounds bearing the INCI name Acrylates/C₁₀₋₃₀ Alkyl Methacrylate Crosspolymer.

5. The cosmetic agent according to any one of the preceding items, characterized in that the percentage by weight of copolymer b), relative to the total weight of the cosmetic agent, is about 0.2 to about 4.0% by weight, preferably about 0.4 to about 3.0% by weight, and in particular about 0.8 to about 2.0% by weight.

6. The cosmetic agent according to any one of the preceding items, characterized in that the polyurethane/polyacrylate hybrid polymer is used in the form of an aqueous dispersion.

7. The cosmetic agent according to any one of the preceding items, characterized in that the polyurethane is a copolymer of the monomers hexylene glycol, neopentyl glycol, adipic acid, methylene diphenyl diisocyanate and dimethylpropionic acid.

8. The cosmetic agent according to any one of the preceding items, characterized in that the polyacrylate is polymethyl methacrylate.

9. The cosmetic agent according to any one of the preceding items, wherein the polyurethane/polyacrylate hybrid polymer b) bears the INCI name Polyurethane-2 and Polymethyl Methacrylate.

10. The cosmetic agent according to any one of the preceding items, characterized in that the weight ratio of copolymer a) to hybrid polymer b) is about 1:1 to about 1:10, preferably about 1:2 to about 1:8, and in particular about 1:4 to about 1:6.

11. The cosmetic agent according to any one of the preceding items, characterized by furthermore comprising, based on the total weight thereof, c) about 1.0 to about 10% by weight polyvinylpyrrolidone and/or vinylpyrrolidone/vinyl acetate copolymer, preferably polyvinylpyrrolidone.

12. The cosmetic agent according to item 9, characterized in that the percentage by weight of the polyvinylpyrrolidone and/or vinylpyrrolidone/vinyl acetate copolymer c), relative to the total weight of the cosmetic agent, is about 2.0 to about 8.5% by weight, and preferably about 3.0 to about 7.0% by weight.

13. The cosmetic agent according to any one of the preceding items, characterized in that the cosmetic agent, based on the total weight thereof, comprises about 0.01 to about 0.2% by weight, preferably about 0.01 to about 0.1% by weight, and in particular about 0.02 to about 0.6% by weight of an amino alcohol, and preferably 2-amino-2-methylpropanol.

14. The cosmetic agent according to any one of the preceding items, characterized in that the cosmetic agent, based on the total weight thereof, comprises at least about 10% by weight, in particular at least about 20.0% by weight, more preferably at least about 40% by weight, and likewise preferably at least about 70% by weight.

16. The cosmetic agent according to any one of the preceding items, characterized in that the agent is present in the form of hair gel, hair spray, hair mousse or hair wax.

17. Use of a cosmetic agent according to any one of items 1 to 16 for temporarily reshaping keratin-containing fibers, and in particular human hair.

18. A method for temporarily reshaping keratin-containing fibers, and in particular human hair, in which a cosmetic agent according to any one of items 1 to 16 is applied to the keratin fibers, and the shape of these is temporarily imparted hold.

### EXAMPLES

The following styling agent was produced:

| | % by | | |
|---|---|---|---|
| Component/raw material | weight | INCI name | Manufacturer |
| Water, demineralized | 96.955 | | |
| Hybridur ® 875 polymer | 2.5 | Polyurethane-2 | Air Products and |
| dispersion | | and Polymethyl | Chemicals, Inc. |
| | | Methacrylate | |
| Carbopol ® Ultrez 21 | 0.5 | Acrylates/C10-30 | Lubrizol |
| | | Alkyl Acrylate | |
| | | Crosspolymer | |
| AMP-ULTRA PC 2000 | 0.045 | Aminomethyl | Dow |
| | | Propanol | |
| Total | 100 | | |

The quantity information in the table is provided in % by weight of the respective raw material, based on the total composition.
The solids content of the Polyurethane-2 and Polymethyl Methacrylate was thus 1% by weight, and the solids content of the Acrylates/C₁₀₋₃₀ Alkyl Acrylate Crosspolymer was 0.5% by weight.

The resultant styling agent exhibited extraordinarily good moisture resistance in the high humidity curl retention test (HHCR test). After 6 hours, a value of 55.9 was established in the HHCR test (mean value of a determination based on 10 curled strands of hair). Further required properties such as stiffness, combability and long-lasting hold were also met.

For comparison, HHRC values were also determined for Hybridur® 875 in water at a solids content of 2% by weight, and Carbopol® Ultrez 21 in water at a solids content of 1.0% by weight.
Results:
Hybridur® 875: mean value of 35.2 determined based on 10 curled strands of hair.
Carbopol® Ultrez 21: mean value of 47.6 determined based on 10 curled strands of hair.

This clearly demonstrated a synergistic effect with respect to the moisture resistance for the combination of copolymers a) and b).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims

1. A cosmetic agent for temporarily reshaping keratin fibers, comprising, based on the total weight thereof:
about 0.1 to about 2.0% by weight of at least one hydrophobically modified (meth)acrylic acid copolymer; and
about 0.1 to about 6.0% by weight of at least one polyurethane/polyacrylate hybrid polymer.

2. The cosmetic agent according to claim 1, wherein the percentage by weight of the hydrophobically modified (meth)acrylic acid copolymer, relative to the total weight of the cosmetic agent, is about 0.2 to about 1.5% by weight.

3. The cosmetic agent according to claim 1, wherein the hydrophobically modified (meth)acrylic acid copolymer is obtained by reacting:
at least one monomer (a1) from the group consisting of acrylic acid, methacrylic acid, C₁-C₆ alkyl acrylic acid esters, and C₁-C₆ alkyl methacrylic acid esters with;
at least one monomer (a2) from the group consisting of the C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates.

4. The cosmetic agent according to claim 1, wherein the percentage by weight of the polyurethane/polyacrylate hybrid polymer, relative to the total weight of the cosmetic agent, is about 0.2 to about 4.0% by weight.

5. The cosmetic agent according to claim 1, wherein the polyurethane/polyacrylate hybrid polymer bears the INCI name Polyurethane-2 and Polymethyl Methacrylate.

6. The cosmetic agent according to claim 1, wherein the weight ratio of the hydrophobically modified (meth)acrylic acid copolymer to the polyurethane/polyacrylate hybrid polymer is about 1:1 to about 1:10.

7. The cosmetic agent according to claim 1, wherein the cosmetic agent, based on the total weight thereof, comprises about 0.01 to about 0.2% by weight.

8. The cosmetic agent according to claim 6, wherein the water content of the cosmetic agent, based on the total weight thereof, is at least about 10% by weight.

9. The cosmetic agent according to claim 1 utilized for temporarily reshaping keratin-containing fibers.

10. A method for temporarily reshaping keratin-containing fibers, comprising:
applying to the keratin fibers a cosmetic agent comprising, based on the total weight thereof:
about 0.1 to about 2.0% by weight of at least one hydrophobically modified (meth)acrylic acid copolymer; and
about 0.1 to about 6.0% by weight of at least one polyurethane/polyacrylate hybrid polymer; and
temporarily imparting hold to the shape of the keratin-containing fibers.

11. The cosmetic agent according to claim 1, wherein the percentage by weight of the hydrophobically modified (meth)acrylic acid copolymer, relative to the total weight of the cosmetic agent, is about 0.3 to about 1.0% by weight.

12. The cosmetic agent according to claim 1, wherein the percentage by weight of the hydrophobically modified (meth)acrylic acid copolymer, relative to the total weight of the cosmetic agent, is about 0.3 to about 0.7% by weight.

13. The cosmetic agent according to claim 1, wherein the percentage by weight of the polyurethane/polyacrylate hybrid polymer, relative to the total weight of the cosmetic agent, is about 0.4 to about 3.0% by weight.

14. The cosmetic agent according to claim 1, wherein the percentage by weight of the polyurethane/polyacrylate hybrid polymer, relative to the total weight of the cosmetic agent, is about 0.8 to about 2.0% by weight.

15. The cosmetic agent according to claim 1, wherein the weight ratio of the hydrophobically modified (meth)acrylic acid copolymer to the polyurethane/polyacrylate hybrid polymer is about 1:2 to about 1:8.

15. The cosmetic agent according to claim 1, wherein the weight ratio of the hydrophobically modified (meth)acrylic acid copolymer to the polyurethane/polyacrylate hybrid polymer is about 1:4 to about 1:6.

17. The cosmetic agent according to claim 1, wherein the cosmetic agent, based on the total weight thereof, comprises about 0.01 to about 0.1% by weight of an amino alcohol.

18. The cosmetic agent according to claim 1, wherein the cosmetic agent, based on the total weight thereof, comprises about 0.02 to about 0.6% by weight of an amino alcohol.

19. The cosmetic agent according to claim 1, wherein the cosmetic agent, based on the total weight thereof, comprises about 0.01 to about 0.2% by weight of 2-amino-2-methylpropanol.

20. A cosmetic agent for temporarily reshaping keratin fibers, comprising, based on the total weight thereof:
about 0.1 to about 2.0% by weight of at least one hydrophobically modified (meth)acrylic acid copolymer the hydrophobically modified (meth)acrylic acid copolymer is obtained by reacting:
at least one monomer (a1) from the group consisting of acrylic acid, methacrylic acid, C₁-C₆ alkyl acrylic acid esters, and C₁-C₆ alkyl methacrylic acid esters with;
at least one monomer (a2) from the group consisting of the C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates; and
about 0.1 to about 6.0% by weight of at least one polyurethane/polyacrylate hybrid polymer wherein the polyurethane/polyacrylate hybrid polymer bears the INCI name Polyurethane-2 and Polymethyl Methacrylate; and
wherein the cosmetic agent, based on the total weight thereof, comprises about 0.01 to about 0.2% by weight of 2-amino-2-methylpropanol.
